(19) United States
(12) Patent Application Publication
(10) Pub. No.: US 2017/0000994 A1
(43) Pub. Date: Jan. 5, 2017

(54) A FLUID APPLICATOR SYSTEM AND METHOD

(71) Applicant: Gerald HIPPERSON, Prague (CZ)

(72) Inventor: Gerald HIPPERSON, Prague (CZ)

(21) Appl. No.: 15/125,728

(22) PCT Filed: Mar. 13, 2015
(86) PCT No.: PCT/EP2015/055359

§ 371 Date: Sep. 13, 2016

(30) Foreign Application Priority Data
Mar. 14, 2014 (GB) 1404581.9

(51) Int. Cl. A61M 35/00; B05B 12/12; B05B 13/04; B05B 12/00

(52) U.S. Cl. CPC A61M 35/003; B05B 12/008; B05B 12/126; B05B 13/04; A61M 2205/18; A61M 2205/582; A61M 2205/581; A61M 2205/583; A61M 2205/52

(57) ABSTRACT

A hand applicator system (100) for spraying a human or animal body (10), the system comprising: a hand applicator (110); and a travel speed indicator (120) which indicates the optimum travel speed of the hand applicator over a region of the body, wherein the travel speed indicator emits a signal to an operator of the hand applicator, the signal varying during a spraying procedure and being indicative of the appropriate instantaneous travel speed of the hand applicator over the region of the body adjacent the hand applicator at any time.

The present disclosure relates to a fluid applicator system and method, in particular, to an improved hand applicator system and method.

### BACKGROUND

With conventional hand spray tanning fluid applicators, such as spray guns, it has been found that an operator spraying a tall person tends to move the spray gun faster than when they are spraying a short person. As a result, insufficient tanning fluid is applied to the tall person compared to the shorter person.

Automated tanning booths may address this issue, but such automated tanning booths are expensive and are not portable in contrast to a hand spray gun applicator. In addition, automated tanning booths are less flexible and less reliable than hand spray applicators.

The present disclosure seeks to address these issues.

### STATEMENTS OF INVENTION

According to a first aspect of the present disclosure there is provided a hand applicator system for spraying a human or animal body, the system comprising: a hand applicator; and a travel speed indicator which indicates a desired, e.g. optimum, travel speed of the hand applicator over a region of the body, wherein the travel speed indicator emits a signal to an operator of the hand applicator, the signal varying to indicate to the operator the appropriate travel speed of the hand applicator. The signal may vary during a spraying procedure. The signal may vary according to the size of the user. The signal may indicate the appropriate instantaneous travel speed of the hand applicator over the region of the body adjacent the hand applicator at any time. The spraying procedure may comprise a number of predetermined traverses or strokes across the body. The strokes or traverses may be along predetermined travel paths along certain parts of the body.

Using a hand applicator that provides an indication of the correct travel speed allows an operator to adjust the speed at which the hand applicator travels, thereby ensuring that the correct amount of fluid is applied for the size of the body or body part being sprayed.

The hand applicator system may further comprise a case. The case may be configured to store the hand applicator, e.g. when not in use. The hand applicator may comprise a spray gun. The travel speed indicator may be provided in the case separate from the hand applicator when in use. Alternatively, the travel speed indicator may be provided in or on the hand applicator.

The signal may comprise an audible, visual and/or tactile signal. The signal may comprise a series of discrete sounds, flashes and/or vibrations. A frequency and/or duty cycle of the sounds, flashes and/or vibrations may be indicative of an appropriate travel speed of the hand applicator. The signal may be generated by one or more of a speaker, buzzer, light, display, vibrator or any other device that may emit a signal that may be sensed by the operator.

The hand applicator system may further comprise a storage medium, e.g. an electronically readable storage medium. The storage medium may comprises data for determining the appropriate speed for the hand applicator to travel over the body using a pre-set series of travel paths. For example, the storage medium may comprise the appropriate travel speeds for spraying the body using the pre-set series of travel paths. The storage medium may comprise an RFID card.

The hand applicator may comprise a speed detector configured to measure the speed of the hand applicator. The travel speed indicator may be configured to adjust the signal to indicate whether the hand applicator is being moved too fast or too slowly over the body. The speed detector may comprise an accelerometer, such as a piezoelectric sensor. A signal from the accelerometer may be integrated, e.g. by a processor, to obtain the speed of the hand applicator.

The hand applicator may comprise a location detector configured to measure the location of the hand applicator relative to the body. The travel speed indicator may be configured to adjust the signal to indicate whether the hand applicator is being moved too fast or too slowly over the body for that location. The location detector may comprise an accelerometer, such as a piezoelectric sensor. A signal from the accelerometer may be integrated, e.g. by a processor, to obtain the location of the hand applicator.

According to a second aspect of the present disclosure there is provided a method of using a hand applicator system for spraying a human or animal body, the hand applicator system comprising: a hand applicator; and a travel speed indicator which indicates the desired travel speed of the hand applicator over a region of the body, wherein the method comprises:
emitting a signal to an operator of the hand applicator, and
varying the signal to indicate to the operator the appropriate travel speed of the hand applicator.

### BRIEF DESCRIPTION OF THE DRAWINGS

For a better understanding of the present disclosure, and to show more clearly how it may be carried into effect, reference will now be made, by way of example, to the accompanying drawings, in which:

FIG. 1 shows a schematic of a fluid applicator system according to an example of the present disclosure;

FIG. 2 shows a method for applying fluid to a body according to an example of the present disclosure;

FIG. 3 shows a according to an example of the present disclosure; and

FIG. 4 shows a hand applicator according to the example of the present disclosure.

### DETAILED DESCRIPTION

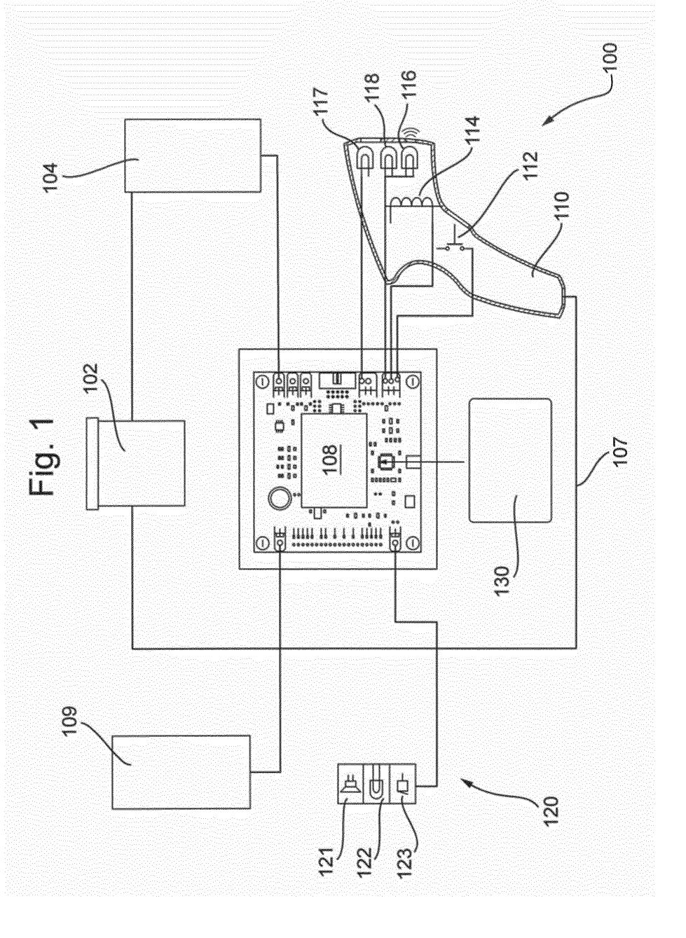

With reference to FIGS. 1 to 4, the present disclosure relates to a hand applicator system 100 for spraying a body 10. The hand applicator system 100 may spray the body 10, such as a human or animal body, with tanning fluid, sun tan lotion, a medicinal fluid or any other fluid or lotion to be applied to the body. The hand applicator system 100 comprises a hand applicator 110, e.g. a spray gun, to generate a spray and apply the fluid to the body. The hand applicator system 100 further comprises a travel speed indicator 120. The travel speed indicator 120 indicates the optimum travel speed of the hand applicator over a region of the body 10 being sprayed.

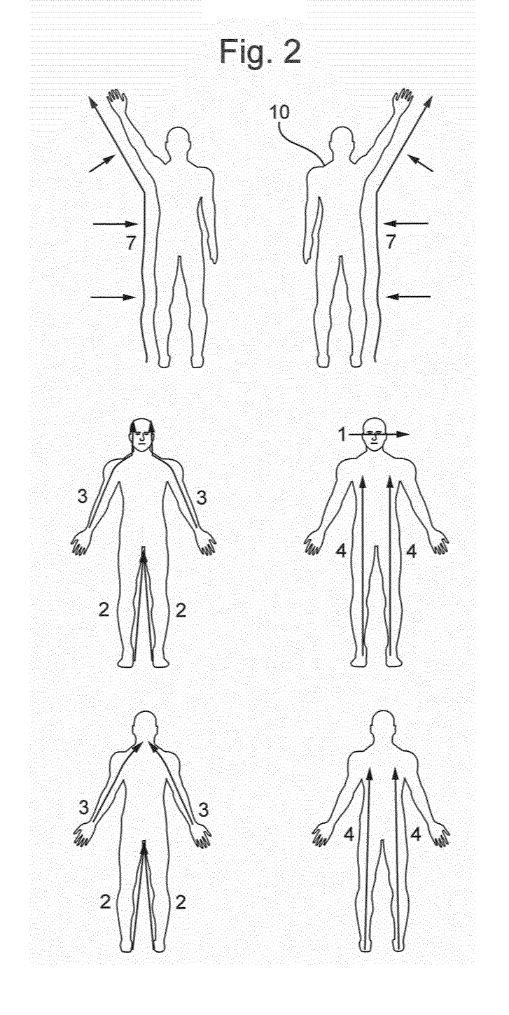

A methodology for spraying the body 10 is depicted in FIG. 2. A pre-set series of travel paths or discrete strokes for the spray applicator 110 are denoted by the arrows shown in FIG. 2. The strokes may traverse the front, back, left hand side, right hand side, across the head and inside the legs of the body. The numbers accompanying each arrow in FIG. 2 indicate the value for each stroke. The value may relate to the duration and/or amount of fluid required for the particular stroke. For example, to apply an even and consistent coat of fluid, the duration of the strokes which cover longer lengths of the body 10 will be longer than for shorter lengths of the body. In particular, the duration of the stroke with value 7 (e.g. the strokes from foot to finger tip) will be longer than the duration of the stroke with value 1 (e.g. the stroke across the face). The travel speed indicator 120 provides a signal that indicates to the operator the required duration of the stroke. This enables the operator to adjust the speed at which they move the hand applicator and overcomes the urge to travel more quickly over longer distances.

Returning to FIG. 1, the hand applicator system 100 comprises a control unit 102 for controlling the spray of fluid. The control unit 102 may be powered by a power source 109. The hand applicator 110 may comprise a trigger 112, which when activated sends an activation signal to the control unit 102. Upon receiving the activation signal, the control unit 102 may send a return signal to activate a valve (not shown) in the hand applicator. When opened, the valve may permit a spray of fluid to be emitted from the hand applicator 110. By way of example, the valve may be selectively opened by a solenoid 114 in the hand applicator 110.

The control unit 102 may also activate a pressure source 104, such as a pump, which pressurises fluid in a tank 106. The tank 106 is in flow communication with the hand applicator via a pipe 107. The pressure source 104 may be activated upon receiving the activation signal from the trigger 112 and/or when the control unit detects that the pressure in the tank 106 is below a predetermined threshold pressure.

The control unit 102 may also receive signals from a proximity sensor 116, which may be provided on the hand applicator 110. The proximity sensor 116 may sense the distance between the hand applicator 110 and the portion of the body to be sprayed. The proximity sensor 116 may emit and sense sound or radiation, such as infrared, to deduce this distance. The distance between a spray gun nozzle 115 and the target is intended to stay the same as much as possible, e.g. to ensure an even coverage of spray, so the optional proximity sensor is available to advise the operator of any significant error in gun-to-target distance. The control unit 102 may advise the operator via an audible, visual and/or tactile signal that the hand applicator 110 is too far or too close to the target.

The hand applicator 110 may comprise a guide light 117, such as a laser, which indicates to the operator where to point the hand applicator 110, e.g. to centre the spray on the portion of the body to be sprayed. Additionally or alternatively, the hand applicator 110 may comprise a light 118, such as an ultra bright LED, which may illuminate the portion of the body being sprayed so as to eliminate any shadows and assist the operator in obtaining an even coverage.

The control unit 102 may control the travel speed indicator 120, e.g. by sending a signal to the travel speed indicator based on the desired travel speed for a particular stroke of the hand applicator. The travel speed indicator 120 is configured to emit a speed signal to an operator of the hand applicator 110. The speed signal varies during a spraying procedure to indicate the appropriate travel speed of the hand applicator over the region of the body adjacent the hand applicator at any time. The speed signal may vary from stroke to stroke. The speed signal may also vary during each stroke.

The travel speed indicator 120 may comprise a sound emitting device 121, such as a speaker or buzzer; a light emitting device 122, such as a lamp or LED; and/or a motion generating device 123, such as a vibrator. Accordingly, the speed signal from the travel speed indicator 120 may comprise an audible, visual and/or tactile signal and the signal may comprise a series of discrete sounds, flashes and/or movements. The sound, light and/or motion emitting devices 121, 122, 123 may be connected to the control unit 102, so that they may be selectively activated by the control unit. Furthermore, a frequency and/or duty cycle of the sounds, lights and/or movements may be controlled by the control unit 102 and these may be indicative of an appropriate travel speed of the hand applicator.

By way of example, the travel speed indicator 120 may comprise a plurality of lights and the lights may flash sequentially to indicate the appropriate travel speed. The lights may be disposed in a line and be illuminated sequentially from one end of the line to the other. The speed at which the illumination moves from one light to the next may be indicative of the appropriate travel speed of the hand applicator. The portion of the spray gun facing the operator in use of the spray gun may be provided with the plurality of lights.

The hand applicator system 100 may further comprise a storage medium 130, which may be electronically readable. The storage medium 130 may store user related data, the user being the person being sprayed by the operator, and the storage medium 130 may be specific to that particular user. By way of example, the storage medium 130 may store: user body dimensions, such as height, waist, chest, inside leg and/or arm measurements; user sex; user name; user account details; and/or other user details. The detailed size measurements of the user, such as inside leg, arm measurements etc. may be stored on the storage medium 130 or they may be estimated by the control unit 102 using just the height and/or weight of the user.

Data from the storage medium 130 may be transferred to the control unit 102. As depicted in FIG. 1, the storage medium 130 may be received in a slot 108, which may be associated with the control unit 102. By way of example, the storage medium 130 may comprise an RFID card, but it may alternatively comprise a card with a magnetic strip, a microchip, punched holes or any other readable storage means. Alternatively or additionally, the user specific data may be manually input, e.g. via an interface of the control unit 102.

The control unit 102 may use the manually entered data or data stored on the storage medium 130 to calculate the appropriate speeds of the hand applicator 110 for each stroke of the hand applicator over a pre-set series of travel paths, e.g. as shown in FIG. 2. Moving the hand applicator 110 at the appropriate speeds may ensure that the correct dosage per surface area of the user's skin is applied. Alternatively, the storage medium 130 may store the appropriate travel speeds for each of the pre-set series of travel paths for the hand applicator over the body.

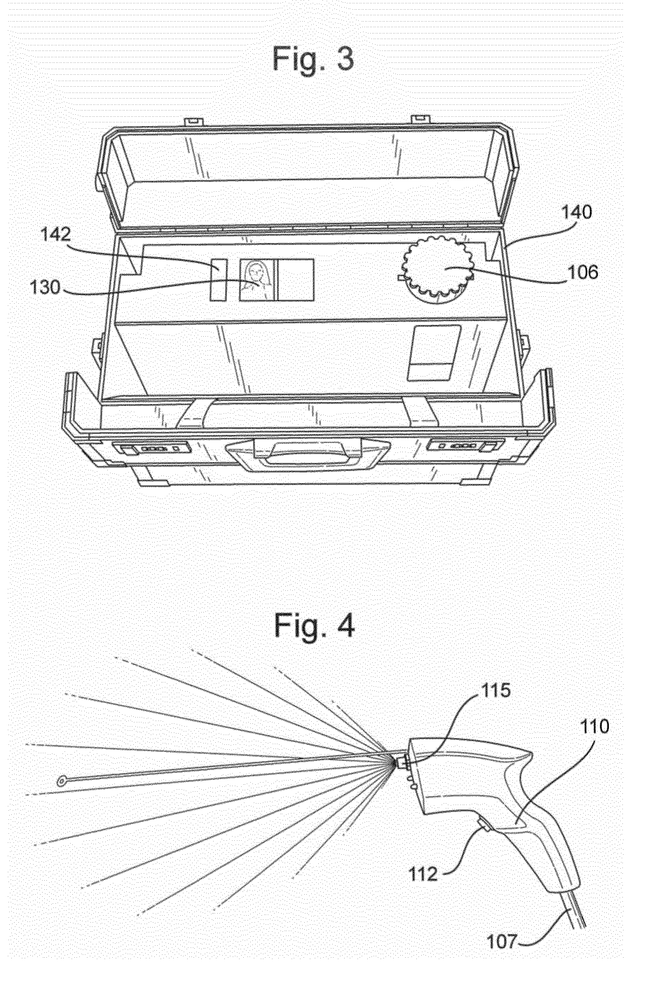

As depicted in FIG. 3, the hand applicator system 100 may further comprise a case 140. The case 140 may be configured to store the hand applicator 110, e.g. when the hand applicator is not being used. The case 140 may be portable. The travel speed indicator 120, e.g. the sound, light and/or motion emitting devices 121, 122, 123, may be provided in the case 140 such that the speed signal is emitted from the case 130. Alternatively, the travel speed indicator 120 may be provided in or on the hand applicator 110. The control unit 102, pressure source 104, tank 106 and/or power source 109 may otherwise be provided in the case 140.

The control unit 102 may interface with a display 142 which may display data relating to the spraying of the body. For example, the display 142 may display one or more of how many strokes of the hand applicator 110 have been performed, which stroke is currently being performed; which stroke is next to be performed, how much liquid has been used, how much liquid remains, and how much liquid was sprayed per stroke. The display 142 may also display user related data, such as user body dimensions, user name etc. As depicted in FIG. 3, the display may be provided in or on the case 140. However, a further or alternative display may be provided on the hand applicator 110.

The hand applicator 110 may comprise a speed detector (not shown) configured to measure the speed of the hand applicator. The travel speed indicator 120 may be configured to adjust the speed signal or provide a further signal to indicate whether the hand applicator is being moved too fast or too slowly over the body. The speed detector may comprise an accelerometer, such as a piezoelectric sensor. A signal from the accelerometer may be integrated, e.g. by the control unit 102, to obtain the speed of the hand applicator 110. The speed detector may provide feedback to the control unit to adjust the signal emitted by the travel speed indicator 120.

Additionally or alternatively, the hand applicator 110 may comprise a location detector (not shown) configured to measure the location of the hand applicator relative to the body 10. The travel speed indicator 120 may be configured to adjust the signal to indicate whether the hand applicator is being moved too fast or too slowly over the body for that location, e.g. body part. The location detector may comprise an accelerometer, such as a piezoelectric sensor. A signal from the accelerometer may be integrated, e.g. by the control unit 102, to obtain the location of the hand applicator. The location detector may provide feedback to the control unit to adjust the signal emitted by the travel speed indicator 120.

In use, the travel speed indicator 120 may emit a particular signal for a particular stroke across the user's body. For example, a low frequency emission may indicate to the operator that the hand applicator is to be moved over a longer period of time for a particular stroke, such as the long stroke from toe to finger tip. By contrast, a high frequency emission may indicate to the operator that the hand applicator is to be moved over a shorter period of time for a particular stroke, such as a short stroke across the face.

In particular, a certain number of emissions, e.g. pulses of light, sound or vibration, from the travel speed indicator 120 may be emitted for each stroke across the user's body. Knowing the number of pulses, the operator can then moderate the speed of the hand applicator to ensure that all of the pulses are emitted during the particular stroke. For example, the operator may count the pulses and knowing the total number of pulses, may moderate the speed of the hand applicator accordingly.

The control unit 102 may vary the frequency of the pulses to suit the particular stroke and its length. The control unit 102 knows the length of the stroke from knowing the dimensions of the user and which stroke is currently being performed.

The frequency of the signal from the travel speed indicator 120 may be constant during a particular stroke across the body. However, it is also envisaged that the signal, e.g. frequency, may vary during a particular stroke to indicate to the operator to speed up or slow down. For example, a high frequency signal may indicate to the operator to speed up the traverse of the hand applicator. By contrast, a low frequency may indicate to the operator to slow down the traverse of the hand applicator. Such variations in the frequency may allow for the hand applicator to pass more quickly over body parts where less fluid may be required, such as an ankle, and by contrast more slowly over body parts where more fluid may be required, such as a thigh. In particular, a certain number of pulses may be emitted over a particular stroke and the frequency at which these pulses may be emitted may vary during the stroke to account for variations in body shape along the stroke.

Once a particular stroke has finished, the control unit 102 may indicate to the operator which stroke is next. The travel speed indicator 120 may indicate to the operator the travel speed required for the next stroke before actually carrying out the stroke. In this way, the operator has an indication of the speed and duration of the next stroke before carrying it out. Once the trigger 112 has been depressed the travel speed indicator 120 may indicate to the operator the travel speed required to guide the operator as they are carrying out the stroke.

Although the description above refers to the spraying of a human or animal body, e.g. with tanning fluid, the system disclosed herein may also apply to other spraying applications where the speed of the hand applicator spray gun is important. For example, the system disclosed herein may be used for spraying car body panels or any other parts by hand.

It will be appreciated by those skilled in the art that although the invention has been described by way of example with reference to one or more examples, it is not limited to the disclosed examples and that alternative examples could be constructed without departing from the scope of the invention as defined by the appended claims.

1. A hand applicator system for spraying at least one of a human and animal body, the system comprising:
a hand applicator; and
a travel speed indicator which indicates the desired travel speed of the hand applicator over a region of the body,
wherein the travel speed indicator emits a signal to an operator of the hand applicator, the signal varying to indicate to the operator the appropriate travel speed of the hand applicator.

2. The hand applicator system of claim 1, wherein the signal varies during a spraying procedure.

3. The hand applicator system of claim 1, wherein the signal varies according to the size of the body being sprayed.

4. The hand applicator system of any of the preceding claims claim 1, wherein the hand applicator system further comprises a case.

5. The hand applicator system of claim 4, wherein the case is configured to store the hand applicator when not in use.

6. The hand applicator system of claim 4, wherein the travel speed indicator is provided in the case separate from the hand applicator when in use.

7. The hand applicator system of claim 1, wherein the travel speed indicator is provided in or on the hand applicator.

8. The hand applicator system of claim 1, wherein the signal comprises an audible, visual and/or tactile signal.

9. The hand applicator system of claim 1, wherein the signal comprises a series of discrete sounds, flashes and/or vibrations.

10. The hand applicator system of claim 9, wherein a frequency and/or duty cycle of the sounds, flashes and/or vibrations is indicative of the appropriate travel speed of the hand applicator.

11. The hand applicator system of claim 1, wherein the hand applicator comprises a spray gun.

12. The hand applicator system of claim 1, wherein the hand applicator system further comprises a storage medium for storing user data.

13. The hand applicator system of claim 12, wherein the storage medium comprises data for determining the appropriate speed for the hand applicator to travel over the body using a pre-set series of travel paths.

14. The hand applicator system of claim 12, wherein the storage medium comprises an RFID card.

15. The hand applicator system of claim 1, wherein the hand applicator comprises a speed detector configured to measure the speed of the hand applicator, and the travel speed indicator is configured to adjust the signal to indicate whether the hand applicator is being moved at least one of too fast and too slowly over the body.

16. The hand applicator system of claim 1, wherein the hand applicator comprises a location detector configured to measure the location of the hand applicator relative to the body, the travel speed indicator being configured to adjust the signal to indicate whether the hand applicator is being moved at least one of too fast and too slowly over the body for that location.

17. A method of using a hand applicator system for spraying at least one of a human and animal body, the hand applicator system comprising:
a hand applicator; and
a travel speed indicator which indicates the desired travel speed of the hand applicator over a region of the body,
wherein the method comprises:
emitting a signal to an operator of the hand applicator, and varying the signal to indicate to the operator the appropriate travel speed of the hand applicator.

18. (canceled)

19. (canceled)
